(19) United States
(12) Patent Application Publication
(10) Pub. No.: US 2009/0110642 A1
(43) Pub. Date: Apr. 30, 2009

(54) METHOD FOR THE PRODUCTION OF BIO-IMAGING NANOPARTICLES WITH HIGH YIELD BY EARLY INTRODUCTION OF IRREGULAR STRUCTURE

(76) Inventors: Kyoungja Woo, Seoul (KR); Jihyung Moon, Seoul (KR)

Correspondence Address: JONES DAY, 222 EAST 41ST ST, NEW YORK, NY, 10017, US

(21) Appl. No.: 11/967,385

(22) Filed: Dec. 31, 2007

(30) Foreign Application Priority Data
Oct. 31, 2007 (KR) KR 10-20070110333

(51) Int. Cl. A61K 49/18; B82B 1/00

(52) U.S. Cl. 424/9.32; 424/9.3; 424/9.1; 977/774

(57) ABSTRACT

Methods of preparing bio-imaging nanoparticles having high dispersibility in an aqueous solution, biocompatibility, and targetability with high yield, by early introduction of an irregular structure are disclosed.

### CROSS REFERENCE TO RELATED APPLICATIONS

This application claims priority to Korean Patent Application No. 10-2007-110333, filed Oct. 31, 2007. The entire contents of that application are hereby incorporated by reference.

### FIELD OF THE INVENTION

The present invention relates to a method of preparing bio-imaging nanoparticles having high dispersibility in an aqueous solution, biocompatibility, and targetability with high yield by early introduction of an irregular surface structure.

### BACKGROUND OF THE INVENTION

Since the establishment of a method of chemically synthesizing hydrophobic inorganic nanoparticles having homogeneous size distribution in an organic solvent including a surfactant, various attempts have been made to put the method to practical use. In particular, since the nanoparticles prepared in an aqueous solution show a much more heterogeneous size distribution than those prepared in an organic solvent and water is the cheapest, most environmentally friendly, and most useful solvent existing on earth, modifying the surface of the hydrophobic nanoparticles having a homogeneous size distribution prepared in an organic solvent so as to be stably dispersed in an aqueous solution is very important and has been a major area of attention for researchers. Soluble quantum dots or soluble nanoparticles containing only a single quantum dot or a single inorganic nanoparticle at the center thereof and bonding organic materials having useful functional groups at the surface thereof can be effectively used as bio-imaging materials as well as basic materials for manufacturing a certain type of nanostructure, such as a biosensor or a memory device. Therefore, research on surface modification of these nanoparticles has been carried out intensively.

The commonly used method for such surface modification comprises the following steps: reacting hydrophobic nanoparticles with an excessive amount of organic ligands containing a thiol (—SH) group and a hydrophilic group being linked by a hydrocarbon chain, thereby replacing all surfactant ligands on the surface of nanoparticles with metal-thiolate (M-S) bonds and exposing the hydrophilic groups outwardly, resulting in hydrophilic nanoparticles; and forming a covalent bond between the hydrophilic group of the nanoparticle and a functional molecule, such as a targeting biomolecule, to obtain bio-imaging nanoparticles including only inorganic nanoparticles at the center thereof (see FIG. 1). Such a covalent bond is composed of an amide bond or an ester bond between the hydrophilic group of the nanoparticle and the new functional molecule. A major subject of study is a polar organic ligand in which a hydrophilic group selected from the group consisting of amine (NH₂), carboxylic acid (COOH), thiol, and hydroxy (OH) is linked to a thiol group via a hydrocarbon chain. It has been well-known that these organic ligands easily form a metal-thiolate (M-S) bond with quantum dots (e.g., CdSe, ZnS, or core/shell CdSe/CdS, CdSe/ZnS, and the like), noble metal nanoparticles (e.g., Au, Ag), or iron oxide magnetic nanoparticles, all of which are characterized by containing abundant metal ingredients on the surface. However, because the hydroxy group or amine group easily aggregates and precipitates in a neutral or near neutral solution, no further studies have been made. On the other hand, since the carboxylic acid group, to a large extent, exists in an ionized state in a neutral solution, thereby showing high dispersibility and stability in solution, it has been widely used as a hydrophilic group for coupling functional molecules to nanoparticles by an amide bond.

However, the above method must go through the step of activating the carboxylic acid groups on the surface of nanoparticles in a weak acidic aqueous solution, which causes the aggregation and precipitation of numerous nanoparticles (W C Chan and S Nie, Science 281: 2016, 1998; Wen Jiang, et al., Chem. Mater. 18: 872, 2006). In particular, the aggregation and precipitation are more severe in the case of magnetic nanoparticles. It is difficult for such nanoparticles that have been aggregated and precipitated to bond to the functional molecules in the next step and, even if such bonding to the functional molecules proceeds, the functional molecules only bond to the surface of aggregated nanoparticles (see step B of FIG. 1, right panel). Such prepared nanoparticles are too big to migrate along the blood vessels and show significantly reduced dispersibility. Further, in the case of using quantum dots, the fluorescence of the nanoparticles is remarkably decreased due to self quenching. Since these precipitates must eventually be removed and only the well-dispersed portion of the solution layer is used, there is a serious problem in that a large quantity of nanoparticles is lost.

In order to overcome the problem of aggregation and precipitation, a method of preparing organic/inorganic complex nanoparticles having a metal-thiolate (M-S) bond by directly reacting hydrophobic inorganic nanoparticles with polyethylene glycol (PEG) having a thiol group has been reported (U.S. Pat. No. 7,041,371). However, the disclosed method still suffers from the problem of low reaction yield, since the hydrophilic thiol group that is linked by a long hydrocarbon chain has to penetrate into the surface of nanoparticles surrounded with surfactants.

In attempting to overcome the problem in the prior art of hydrophilic nanoparticles aggregating and being precipitated, the present inventors have found that such aggregation and precipitation are caused by the hydrogen bonding attraction due to the excessive amount of hydrophilic groups present on the surface of hydrophilic nanoparticles having an uniform structure. On the basis of the above finding, the present inventors have developed a method of structurally hindering such hydrogen bonding attractions and securing the independence and individuality of the nanoparticles during the entire reaction process. The method of the present invention can prepare bio-imaging nanoparticles that contain a single particle at the center thereof without causing aggregation and precipitation and show excellent physical properties, such as homogeneous size distribution, high dispersibility and stability in solution, biocompatibility, targetability and the like.

### BRIEF DESCRIPTION OF DRAWINGS

The embodiments of the present invention will be described in detail with reference to the following drawings.

FIG. 1 depicts the conventional process for preparing bio-imaging nanoparticles, as well as the structure of the nanoparticles obtained in each step.

FIG. 2 depicts the process for preparing bio-imaging nanoparticles according to the present invention, as well as the structure of the nanoparticles obtained in each step.

FIG. 3 shows infrared spectra of quantum dots used as a starting material and quantum dots prepared in Examples 1 to 5 of the present application. a) CdSe/CdS-ODA quantum dots used as a starting material; b) CdSe/CdS-DA quantum dots prepared in Example 1; c) CdSe/CdS(-DA)ₑₓ(-MUA)₅ quantum dots prepared in Example 2; d) CdSe/CdS(-DA)ₑₓ(-MUA-en-FA)₅ quantum dots prepared in Example 3; e) CdSe/CdS(-MPA)ₑₓ-(-MUA-aPEGa)₅ quantum dots prepared in Example 4; f) CdSe/CdS(-MPA)ₑₓ(-MUA-aPEGa-FA)₅ quantum dots prepared in Example 5.

FIG. 4 shows transmission electron microscope (TEM) images of quantum dots used as a starting material and quantum dots prepared in Examples 1 to 5 of the present application. a) CdSe/CdS-ODA quantum dots used as a starting material; b) CdSe/CdS-DA quantum dots prepared in Example 1; c) CdSe/CdS(-DA)ₑₓ(-MUA)₅ quantum dots prepared in Example 2; d) CdSe/CdS(-DA)ₑₓ(-MUA-en-FA)₅ quantum dots prepared in Example 3; e) CdSe/CdS(-MPA)ₑₓ(-MUA-aPEGa)₅ quantum dots prepared in Example 4; f) CdSe/CdS(-MPA)ₑₓ(-MUA-aPEGa-FA)₅ quantum dots prepared in Example 5.

FIG. 5 shows fluorescence microscope images of HT1080 cells and KB cells treated with quantum dots prepared in Examples 4 and 5 of the present application used as a control agent and targeting agent, respectively. QD: CdSe/CdS(-MPA)ₑₓ(-MUA-aPEGa)₅ quantum dots used as a control; QD-FA: CdSe/CdS(-MPA)ₑₓ(-MUA-aPEGa-FA)₅ quantum dots used as a targeting agent; +FA: the presence of excess free FA; −FA: the absence of free FA.

FIG. 6 shows infrared spectra of quantum dots prepared in Example 6 of the present application. a) CdSe/CdS(-DA)ₑₓ(-MUA-aPEGa)₅ quantum dots; b) CdSe/CdS(-DA)ₑₓ(-MUA-aPEGa)₁₀ quantum dots; c) CdSe/CdS(-DA)ₑₓ(-MUA-aPEGa)₃₀ quantum dots.

FIG. 7 shows TEM images of quantum dots prepared in Example 6 of the present application. a) CdSe/CdS(-DA)ₑₓ(-MUA-aPEGa)₅ quantum dots; b) CdSe/CdS(-DA)ₑₓ(-MUA-aPEGa)₁₀ quantum dots.

FIG. 8 shows infrared spectra of magnetic nanoparticles used as a starting material and magnetic nanoparticles prepared in Examples 7 to 11 of the present application. a) SPION-OA nanoparticles used as a starting material; b) SPION(-OA)ₑₓ(MHA)₁₀ nanoparticles prepared in Example 7; c) SPION(-OA)ₑₓ(MHA-aPEGa-MTX)₁₀ nanoparticles prepared in Example 8; d) SPION(-MPA)ₑₓ(MHA-aPEGa-MTX)₁₀ nanoparticles prepared in Example 9; e) SPION(-Lys)ₑₓ(MHA-aPEGa-MTX)₁₀ nanoparticles prepared in Example 10; f) SPION(-OA)ₑₓ(MHA-en-FA)₅ nanoparticles prepared in Example 11.

FIG. 9 shows TEM images of magnetic nanoparticles used as a starting material and magnetic nanoparticles prepared in Examples 7 to 10 of the present application. a) SPION-OA nanoparticles used as a starting material; b) SPION(-OA)ₑₓ(MHA)₁₀ nanoparticles prepared in Example 7; c) SPION(-OA)ₑₓ(MHA-aPEGa-MTX)₁₀ nanoparticles prepared in Example 8; d) SPION(-MPA)ₑₓ(MHA-aPEGa-MTX)₁₀ nanoparticles prepared in Example 9; e) SPION(-Lys)ₑₓ(MHA-aPEGa-MTX)₁₀ nanoparticles prepared in Example 10.

FIG. 10 shows magnetic resonance (MR) images of KB cells targeted with SPION(-OA)ₑₓ(MHA-en-FA)₅ magnetic nanoparticles prepared in Example 11 of the present application.

### SUMMARY OF THE INVENTION

An object of the present invention is to provide a method of preparing bio-imaging nanoparticles by early introduction of an irregular structure into the surface of nanoparticles via partial surface modification, allowing the structural hinderance of the aggregation and precipitation phenomena due to internanoparticle hydrogen bonding attraction, thereby preparing bio-imaging nanoparticles having high dispersibility and stability in a nonpolar organic solvent or an aqueous solution with high yield without the loss of nanoparticles.

It is another object of the present invention to provide bio-imaging nanoparticles having high dispersibility and stability prepared according to the method of the present invention.

The present invention provides a method of preparing bio-imaging nanoparticles which comprises:
1) adding 1 to 30 equivalents of an organic ligand which contains a thiol group and a hydrophilic group being linked by a hydrocarbon chain of from 8 to 20 carbon atoms to core or core/shell structured hydrophobic inorganic nanoparticles protected with surfactants, thereby partially replacing the surfactants with the organic ligands and forming a metal-thiolate (M-S) bond on the surface of nanoparticles, which results in the production of hydrophobic nanoparticles whose surface is only partially modified to be hydrophilic, while still maintaining their individual dispersibility in a nonpolar organic solvent;
2) bonding functional molecules to the hydrophilic groups introduced onto the surface of nanoparticles prepared in step 1), to afford functionality and an irregular structure to the surface of nanoparticles while maintaining their individual dispersibility; and
3) replacing the rest of the surfactants remaining on the surface of nanoparticles prepared in step 2) with organic ligands where at least two hydrophilic groups are linked by a hydrocarbon chain of from 1 to 7 carbon atoms, thereby converting the hydrophobic nanoparticles into hydrophilic ones.

Further, the present invention provides bio-imaging nanoparticles prepared according to the method of the present invention, which are partially hydrophilic only at the portion where organic ligands containing a thiol group and a hydrophilic group being linked by a hydrocarbon chain of from 8 to 20 carbon atoms are introduced into the surface of core or core/shell hydrophobic nanoparticles protected with surfactants, but are still hydrophobic as a whole and maintain their individual dispersibility in a nonpolar organic solvent.

The present invention also provides bio-imaging nanoparticles prepared according to the method of the present invention, which have functionality and an irregular structure owing to the bonding of the hydrophilic groups introduced into the surface of nanoparticles to functional molecules, and are partially hydrophilic only at the portion where the functional molecules are bound to but are still hydrophobic as a whole.

Finally, the present invention provides bio-imaging nanoparticles prepared according to the method of the present invention, which are wholly hydrophilic by replacing the rest of the surfactants remaining on the surface of the nanoparticles with organic ligands having at least two hydrophilic groups being linked by a hydrocarbon chain of from 1 to 7 carbon atoms and maintain their individual dispersibility in an aqueous solution.

### DETAILED DESCRIPTION OF THE INVENTION

The conventional surface modification method of preparing bio-imaging nanoparticles resulted in a significant reduction of production yield, because of the aggregation and precipitation of hydrophilic nanoparticles, prepared by introducing hydrophilic organic ligands into the surface thereof, due to hydrogen bonding attraction which is caused by the uniform structure of the introduced surface hydrophilic groups as themselves in an aqueous solution or in the process of bonding reaction with biomolecules.

The present inventors have found that such aggregation and precipitation of the hydrophilic nanoparticles having a homogeneous size distribution in an aqueous solution is due to the internanoparticle hydrogen bonding attraction caused by the uniform structure of the hydrophilic organic ligands. On the basis of the above finding, the present inventors have developed a method of preparing bio-imaging nanoparticles independently dispersed in an aqueous solution with high yield, which is characterized by introducing early an irregular structure of functional organic materials, such as biocompatible molecules, targeting molecules, a complex or a mixture thereof, into the surface of nanoparticle via a gradual surface modification, thereby preparing hydrophobic nanoparticles showing biocompatibility and/or targetability; and then converting the hydrophobic nanoparticles into hydrophilic ones, which results in suppressing the internanoparticle hydrogen bonding attraction due to the steric hindrance of the functional organic ligands.

In particular, the method of preparing bio-imaging nanoparticles according to the present invention comprises the steps of:
1) adding 1 to 30 equivalents of an organic ligand which contains a thiol group and a hydrophilic group being linked by a hydrocarbon chain of from 8 to 20 carbon atoms to core or core/shell structured hydrophobic inorganic nanoparticles protected with surfactants, thereby partially replacing the surfactants with the organic ligands and forming a metal-thiolate (M-S) bond on the surface of nanoparticles, which results in the production of hydrophobic nanoparticles whose surface is only partially modified to be hydrophilic, while still maintaining their individual dispersibility in a nonpolar organic solvent;
2) bonding functional molecules to the hydrophilic groups introduced onto the surface of nanoparticles prepared in step 1), to afford functionality and an irregular structure to the surface of nanoparticles while maintaining their individual dispersibility; and
3) replacing the rest of the surfactants remaining on the surface of nanoparticles prepared in step 2) with organic ligands where at least two hydrophilic groups are linked by a hydrocarbon chain of from 1 to 7 carbon atoms, thereby converting the hydrophobic nanoparticles into hydrophilic ones.

As mentioned above, the conventional method can secure the hydrophilicity of nanoparticle powders by synthesizing hydrophobic nanoparticles having a homogeneous size distribution in an organic solution containing a surfactant, forming a metal-thiolate (M-S) bond between the nanoparticle and an organic ligand, which contains a thiol group and a hydrophilic group being linked with a short hydrocarbon chain, thereby allowing the hydrophilic functional groups to be exposed outwardly. However, it is extremely easy for these hydrophilic nanoparticles having the outwardly exposed hydrophilic groups to aggregate and be precipitated and, therefore, the nanoparticles cannot successfully form an amide bond or an ester bond with other biomacromolecules. For this reason, there have been no reports that bio-imaging nanoparticles containing a single nanoparticle at the center thereof have been successfully prepared with high yield.

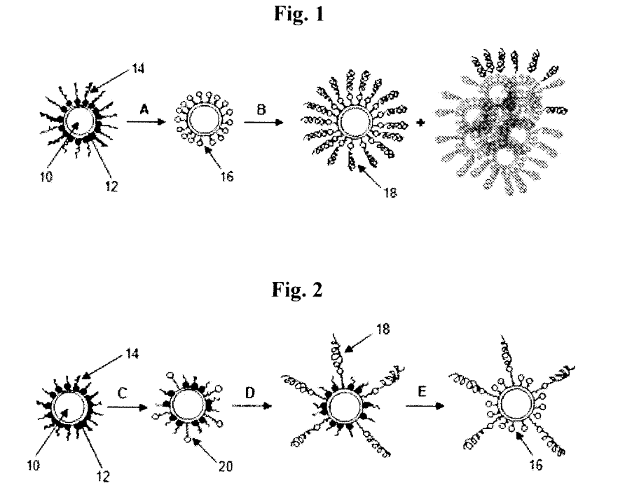

The present inventors have found that various kinds of hydrophilic nanoparticles including hydrophilic nanoparticles having outwardly exposed amine or carboxyl groups, as illustrated in FIG. 1, are easy to aggregate and precipitate due to the internanoparticle hydrogen bonding attraction which is caused by an excessive amount of the hydrophilic functional groups being regularly self-assembled on the surface of nanoparticles. In order to prevent such phenomena, the present invention has established for the first time the optimal conditions for completely suppressing the internanoparticle hydrogen bonding attraction by introducing an irregular structure into the surface of nanoparticles via partial surface modification, as illustrated in FIG. 2. Under those conditions, the nanoparticles are dispersed independently of each other without causing any aggregation or precipitation. First, only a portion of the surfactants existing on the surface of nanoparticles are replaced with organic ligands which contain a thiol group and a hydrophilic group being linked by a hydrocarbon chain of from 8 to 20 carbon atoms, leading to the formation of a metal-thiolate (M-S) bond between them. After that, the hydrophilic groups introduced onto the surface of nanoparticles are coupled with functional molecules having biocompatibility and targetability, imparting the nanoparticles with functionality and an irregular structure. Thus prepared hydrophobic functional nanoparticles can be efficiently used by themselves for the specific purpose of bio-imaging, such as in vitro cell experiments. Subsequently, the rest of the surfactants are replaced with organic ligands which contain at least two hydrophilic groups being linked by a hydrocarbon chain of from 1 to 7 carbon atoms, thereby converting the hydrophobic nanoparticles into hydrophilic ones. As a result, the internanoparticle hydrogen bonding attraction is sterically hindered and, thus, it is possible to prepare bio-imaging nanoparticles containing only a single nanoparticle at the center thereof with high yield and showing improved dispersibility and stability in an aqueous solution.

Referring to FIG. 2, the method of preparing bio-imaging nanoparticles according to the present invention is described in more detail.

In step 1), the hydrophobic nanoparticles are subjected to partial surface modification (see C of FIG. 2). To an organic solution containing inorganic nanoparticles that have core 10 or core/shell 12 structure and are being protected with surfactants 14 was added a small amount of organic ligand 20 which contains a thiol group and a hydrophilic group capable of forming a chemical bond with metal elements on the surface of nanoparticles, where the two groups are linked by a hydrocarbon chain of from 8 to 20 carbon atoms, and then, the mixture is reacted under vigorous stirring. It is preferable to add the organic ligand in the amount of 1 to 30 equivalents. As a result, only a portion of the surfactants are replaced with the organic ligands, and the replaced organic ligands form metal-thiolate (M-S) covalent bonds with metal elements on the surface of the nanoparticles.

In the above step, the inorganic nanoparticles have a core or core/shell structure where their surface, rather than the inside, is stoichiometrically rich in metal elements, and said metal element is capable of chemically binding with the thiol group of the organic ligand via a metal-thiolate (M-S) covalent bond. Preferably, the inorganic nanoparticles are noble metal nanoparticles, iron oxide magnetic nanoparticles, or semiconductor nanoparticles that are composed of one element selected from zinc, cadmium, and lead that belong to family II of the periodic table and one element selected from sulfur, selenium, and tellurium that belong to family VI of the periodic table. More preferably, examples of the inorganic nanoparticles include CdSe, ZnS, CdSe/CdS, CdSe/ZnS, PbS, Au, Ag, Fe₂O₃, Fe₃O₄, and the like, where any nanoparticle composed of materials capable of forming a covalent bond with a thiol group can be used without limitation.

Since the organic ligand being bound to the surface of inorganic nanoparticles contains at least one thiol group and at least one hydrophilic group within the molecule, it can form at least one metal-thiolate (M-S) bond with the inorganic nanoparticle and provide at least one binding site for coupling with other biomolecules 18, such as biocompatible molecules, targeting molecules, a complex (biocompatible-targeting molecules), or a mixture thereof.

Further, the length of the hydrocarbon chain in the organic ligand is preferably in the range from 8 to 20 so as to maintain the nanoparticle's hydrophobicity. If the length of the hydrocarbon chain is less than 8, the nanoparticles are in danger of losing their hydrophobicity. Further, there is the risk of the hydrophilic groups being buried under the surfactants due to their chain length being too short, which may cause a problem in that the buried hydrophilic group is difficult to form a covalent bond with the functional molecule. There is no organic ligand having the length of a hydrocarbon chain exceeding 20 yet, but such a ligand can also be used if it is developed. The amount of the organic ligand added is preferably in the range of from 1 to 30 equivalents. In case of adding 1 equivalent or less, there is the possibility of the nanoparticles remaining in the original state having no replaced organic ligand, causing a reduction in production yield. In the case of adding more than 30 equivalents, the organic ligands are excessively introduced onto the surface of nanoparticles, thereby strengthening the hydrogen bonding attraction between the nanoparticles and causing the aggregation and precipitation of the nanoparticles.

The number of organic ligands which are capable of chemically binding to the surface of inorganic nanoparticles via a metal-thiolate (M-S) covalent bond between the metal element of the nanoparticle and the thiol group of the organic ligand greatly varies depending on the particle size, and may be in the range of from 1 to 150. However, it has been confirmed that if 30 or more M-S bonds are formed and biocompatible molecules bind thereto, the nanoparticles cluster to form an aggregate and are not dispersed in any kind of solvent. Therefore, the number of organic ligands suitable for chemically binding to the surface of inorganic nanoparticles via a metal-thiolate (M-S) covalent bond between the metal element of the nanoparticle and the thiol group of the organic ligand is preferably in the range of from 1 to 30. Suitable examples of organic ligands for forming an irregular structure at the surface of nanoparticles include, but are not limited to, mercaptoundecanoic acid, mercaptododecanoic acid, mercaptohexadecanoic acid, and the like.

Even if the partially surface-modified inorganic nanoparticles in the above step contain the outwardly exposed hydrophilic groups, since the length of the hydrocarbon chain to which the hydrophilic groups are linked is long, they actually do not exhibit hydrophilicity. Further, since the number of the hydrophilic groups is significantly lower than that of the hydrophobic surfactants, these nanoparticles still exhibit hydrophobicity and, thus, are easy to be dispersed in an inorganic solvent.

In step 2), functional molecules, such as biocompatible molecules, targeting molecules, a complex or a mixture thereof, bond to the hydrophilic groups introduced onto the surface of nanoparticles prepared in step 1), thereby producing hydrophobic nanoparticles having an irregular structure. Since thus prepared nanoparticles are still hydrophobic or amphiphilic, they are easy to be dispersed in a nonpolar solvent and can be used for the specific purpose of bioimaging, such as in vitro cell experiments.

In step 3), organic ligand 16 which contains at least two hydrophilic groups being linked by a hydrocarbon chain of from 1 to 7 carbon atoms is added to the nanoparticles prepared in step 2), to replace the rest of the surfactants remaining at the surface of nanoparticles, which results in converting the hydrophobic nanoparticles into hydrophilic ones. Suitable examples of organic ligands in this step include, but are not limited to, mercaptohexanoic acid, mercaptoacetic acid, mercaptopropionic acid, dimercaptosuccinic acid, 2-mercaptoethanol, 2-aminoethanethiol, lysine, arginine, aminovaleric acid, and the like.

The irregular structure introduced into the surface of nanoparticles according to steps 1) and 2) plays an important role in maintaining individual dispersibility by fundamentally preventing the internanoparticle hydrogen bonding which may occur in step 3). Further, the short hydrocarbon chain of from 1 to 7 carbon atoms of the organic ligand introduced in step 3) increases the polarity of the organic matter, thereby improving the dispersibility in water.

The functional molecules used in the present invention are synthetic polymers or bioconstituents that exhibit high biocompatibility without causing side effects and have biodegradability and various physical states, such as solution, gel or membrane, according to their conditions. Since they contain one functional group selected from an amine group, an aldehyde group, and a carboxylic acid group at one or both terminal ends thereof, the functional molecule can form an amide or an ester bond with the hydrophilic group of the nanoparticle. Such functional molecules are exemplified by biocompatible molecules, targeting molecules, a complex (biocompatible-targeting molecules), or a mixture thereof.

It is preferred that the biocompatible molecules suitable for the present invention contain one functional group selected from an amine group, an aldehyde group, and a carboxylic acid group at both terminal ends, or contain one of the same at one terminal end and an alkoxy group or a hydroxy group having 1 to 7 carbon atoms at the other terminal end. Suitable examples of these biocompatible molecules may include, but are not limited to, polyethylene glycol (PEG), dextran, poly(L-lactide) (PLLA), poly(DL-lactide) (PDLLA), poly-DL-lactide/glycolide copolymer (PLGA), chitosan, alginic acid, hyaluronic acid, collagen, heparin, poly(ε-caprolacton), and the like.

Further, the targeting molecules used in the present invention can be specifically recognized in vivo, contain one of an amine group, an aldehyde group, a carboxylic acid group, a hydroxy group, and a thiol group and, thus, can be linked to the organic ligands or biocompatible molecules via one of an amide bond, an ester bond, and a thioester bond. Examples of these targeting molecules may include folic acid, methotrexate (MTX), a peptide selective for a certain cell (e.g., RGD peptide or tat peptide), or an antibody selectively reacting with a certain antigen (e.g., biotin specific for streptavidin, PSA antibody specific for PSA), but are not limited thereto.

Further, the biocompatible molecules and targeting molecules can be used in a complex form prepared by coupling them via an amide bond or an ester bond or in a mixture form thereof.

Since the functional molecules including these biocompatible molecules, targeting molecules, biocompatible-targeting complex, or mixture thereof contain one functional group selected from an amine group, an aldehyde group, and a carboxylic acid group at one or both terminal ends thereof, they can form an amide bond or an ester bond with the carboxylic acid, hydroxy, or amine group of the nanoparticles individually dispersed in step 2), to produce nanoparticles having an irregular structure at the surface thereof.

As described in step B of FIG. 1, since the conventional process for preparing nanoparticles carries out an amide or an ester bonding reaction of hydrophilic nanoparticles in the aggregated form, the functional molecules bond only to the surface of the aggregated nanoparticles and cannot contact the nanoparticles present inside the aggregate, thereby making it impossible to carry out the desired binding reaction. Such problems cause the decrease in the production yield of the bio-imaging nanoparticles and a falling-off in imaging function.

However, as described in step D of FIG. 2, the method of the present invention first prepares hydrophobic functional nanoparticles having an irregular structure by partially replacing the surfactants present at the surface of nanoparticles with organic ligands being linked by a hydrocarbon chain of from 8 to 20 carbon atoms via stepwise partial surface modification, and then, coupling functional molecules to these organic ligands introduced into the surface of nanoparticles. The rest of the surfactants at the surface of the nanoparticles are then replaced with organic ligands being linked by a hydrocarbon chain of from 1 to 7 carbon atoms, converting the hydrophobicity of the nanoparticles into hydrophilicity. According to the method of the present invention, since the aggregation and precipitation of the nanoparticles due to the internanoparticle hydrogen bonding attraction are sterically hindered by the irregular structure introduced onto the surface thereof, it is facile to prepare organic/inorganic complex nanoparticles containing a single nanoparticle at the center thereof with a 100% yield while maintaining their quantum efficiency without the loss of quantum dots.

According to a preferred embodiment of the present invention, hydrophobic, functional bio-imaging nanoparticles having an irregular structure are prepared by sequentially coupling the long chain organic ligands and functional molecules to the nanoparticles via stepwise partial surface modification under conditions of synthesizing core or core/shell nanoparticles or iron oxide magnetic nanoparticles having a round shape of 20 nm or less and fluorescent property in an organic solution and securing the particle's uniformity. Further, the hydrophobicity of the above nanoparticles is converted into hydrophilicity where the steric hindrance from the irregular structure introduced into the surface of nanoparticles prevents the internanoparticle hydrogen bonding attraction, thereby preparing bio-imaging nanoparticles capable of maintaining individual dispersibility in an aqueous solution with a 100% yield.

Further, according to the method of the present invention, as long as the metal element on the surface of nanoparticles can form a covalent bond with the organic ligand having a thiol group, regardless of the constitutional components of the inner inorganic nanoparticles, the nanoparticles are capable of bonding to functional molecules by means of the hydrophilic group of the organic ligand. Therefore, the method of the present invention can be effectively used for preparing organic/inorganic complex nanoparticles containing various kinds of single nanoparticles, as well as quantum dots and magnetic nanoparticles.

In addition, even if the covalent bond between the inorganic nanoparticle and the organic ligand is not a metal-thiolate (M-S) bond, when an amine group, a carboxylic acid group, or a hydroxy group is exposed at the end of the organic ligands introduced into the surface of nanoparticles via stepwise partial surface modification, such a hydrophilic group can bond to the functional molecules, form an irregular structure capable of sterically hindering the internanoparticle hydrogen bonding and then convert the hydrophobic nanoparticles into hydrophilic ones. As a result, the method of the present invention can be effectively applied to prepare various kinds of individually dispersed bio-imaging nanoparticles before and after the conversion into hydrophilic nanoparticles.

Furthermore, the present invention provides bio-imaging nanoparticles containing a single inorganic nanoparticle at the center thereof prepared according to the method of the present invention.

Since the bio-imaging nanoparticles according to the present invention contain organic ligands having a long hydrocarbon chain which are bonded to the part of the inorganic nanoparticle surface having a core or a core/shell structure at the center thereof, the nanoparticles are hydrophilic only at the part where the organic ligands are bonded, but are still mostly hydrophobic in parts where the organic ligands are not bonded (step 1 of the method of the present invention). Further, the present invention can prepare hydrophobic nanoparticles having an irregular surface structure by coupling functional molecules to the hydrophilic groups of said nanoparticles through the formation of an amide bond or an ester bond (step 2 of the method of the present invention). Furthermore, the present invention can provide hydrophilic nanoparticles where the rest of the surfactants remaining on the surface of said nanoparticles are replaced by a short chain of organic ligands having at least two hydrophilic groups, leading to the conversion of their hydrophobicity into hydrophilicity. Thus prepared hydrophilic nanoparticles can maintain their individual dispersibility in an aqueous solution during the entire procedure, because the internanoparticle hydrogen bonding is interrupted by the steric hindrance of the irregular structure introduced into the surface of nanoparticles (step 3 of the method of the present invention).

The hydrophilic bio-imaging nanoparticles of the present invention contain a number of hydrophilic organic ligands capable of forming a hydrogen bond at the surface of a single nanoparticle, but the functional molecules are coupled to the long hydrocarbon chains of the organic ligands at some parts of the nanoparticles, which gives the nanoparticles an irregular structure. Since such an irregular structure exhibits a steric hindrance effect on the formation of hydrogen bonds among the nanoparticles, it is possible to prepare nanoparticles having a uniform size distribution, high chemical stability, dispersibility in an aqueous solution, biocompatibility, targetability, luminosity, and photostability without losing nanoparticles due to aggregation and precipitation.

Therefore, the nanoparticles of the present invention can not only be effectively used as bio-imaging material, but also as basic medical material for high-performance diagnosis and treatment of disease.

The present invention will now be described in detail with reference to the following examples, which are not intended to limit the scope of the present invention.

In the following examples, hydrophobic nanoparticles CdSe/CdS-ODA having a core/shell structure, which are used as a starting material for quantum dots, have octadecylamine (ODA) surfactants coordinated over their surface. According to the methods described in J J Li et al., Journal of the American Chemical Society 125: 12567-12575 (2003) and W W Yu et al., Chemistry of Materials 15: 2854-2860 (2003), Cd, S, Cd, S and Cd were each grown successively, 0.5 layer each (total 2.5 layers), on the surface of the CdSe core nanoparticles.

Further, in the following examples, hydrophobic iron oxide nanoparticles Fe₂O₃—OA, which are used as a starting material for magnetic nanoparticles, have oleic acid (OA) surfactants coordinated over their surface. According to the methods described in K. Woo, et al., Chemistry of Materials 16: 2814-2818 (2004) and K. Woo, et al., IEEE Transactions on magnetics 41, 4137-4139 (2005), Fe₂O₃ or Fe₃O₄ nanoparticles were synthesized and mixed with 10 mole % of a precursor Fe(CO)₅ in inert atmosphere, thereby preparing magnetic nanoparticles stoichimetrically containing excessive iron ingredients at the surface thereof. However, it is obvious to a person skilled in the art that, regardlesss of the preparation method, if the hydrophobic nanoparticles are stoichimetrically abundant in metal ingredients at their surface, they can be used in Examples 1 to 11 without discrimination.

Further, the terms, (-DA)ₑₓ and (-OA)ₑₓ, in the following Examples is a symbol representing excessive decylamine (DA) or oleic acid (OA) at the surface of nanoparticles. Although some ligands among them are replaced with other ligands having thiol groups, since its reducing effect is insignificant and there still exists excessive DA or OA, they are indiscriminately described as (-DA)ₑₓ or (-OA)ₑₓ.

### EXAMPLE 1

### Preparation of Semiconductor Nanoparticles CdSe/CdS-DA by Surface Ligand Exchange

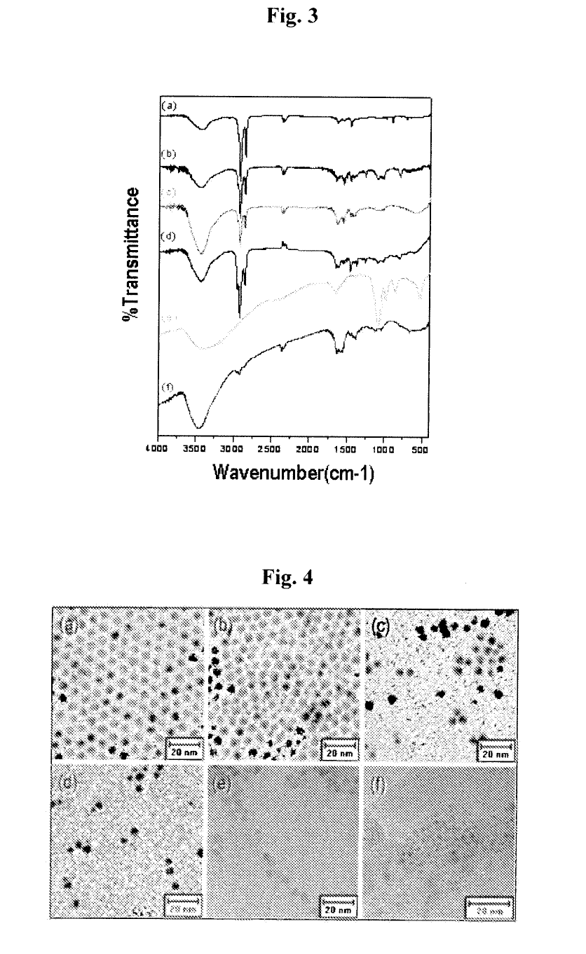

5 ml of a hydrophobic CdSe/CdS-ODA quantum dot solution (8×10⁻⁵ M) was subjected to vacuum evaporation to remove the solvent and dispersed in 20 ml of chloroform. To the dispersion was added 1000 equivalents of decylamine (DA), and the mixture was stirred for 2 days in a dark inert atmosphere. The resulting solution was mixed with acetone and centrifuged to separate the precipitate. Thus separated precipitate was dispersed in chloroform to prepare 20 ml of a CdSe/CdS-DA solution (2×10⁻⁵ M). The CdSe/CdS-DA sample was analyzed with an infrared spectrophotometer and a transmission electron microscope (TEM), where the results are shown in FIG. 3(b) and FIG. 4(b), respectively. The occurrence of surface ligand exchange was confirmed by the shorter distance between CdSe/CdS-DA quantum dots than CdSe/CdS-ODA in the TEM image.

### EXAMPLE 2

### Preparation of Partially Surface Modified Semiconductor Nanoparticles CdSe/CdS(-DA)ₑₓ(-MUA)₅

To 17 ml of the CdSe/CdS-DA solution prepared in Example 1 was added 5 equivalents of mercaptoundecanoic acid (MUA) and stirred for 19 hours in a dark inert atmosphere. The resulting solution was concentrated, mixed with acetone, and centrifuged to separate the precipitate. Thus separated precipitate was dispersed in chloroform to prepare 17 ml of a CdSe/CdS(-DA)ₑₓ(-MUA)₅ solution (2×10⁻⁵ M). The resulting sample was analyzed with an infrared spectrophotometer and a transmission electron microscope (TEM), where the results are shown in FIG. 3(c) and FIG. 4(c), respectively. The TEM image confirmed that self-assembly of the nanoparticles did not occur any longer, due to the destruction of their uniform structure caused by a partial replacement of MUA, as in step C of FIG. 2.

### EXAMPLE 3

### Preparation of Targeting Hydrophobic Semiconductor Nanoparticles CdSe/CdS(-DA)ₑₓ(-MUA-en-FA)₅

2 ml of the CdSe/CdS(-DA)ₑₓ(-MUA)₅ solution prepared in Example 2 was diluted with chloroform to a final volume of 10 ml. After 5 equivalents of dicyclohexylcarbodiimide (DCC) was added to the diluent and stirred for 3 hours in a dark inert atmosphere, 50 equivalents of en-FA prepared as follows were added thereto and further stirred for 2 hours. The resulting solution was concentrated, mixed with acetone, and centrifuged to separate the precipitate. Thus separated precipitate was dispersed in chloroform, to prepare 10 ml of a CdSe/CdS(-DA)ₑₓ(-MUA-en-FA)₅ solution (4×10⁻⁶ M). The bonding of en-FA to MUA was analyzed with an infrared spectrophotometer and a TEM, where the results are shown in FIG. 3(d) and FIG. 4(d), respectively.

Preparation of a Complex en-FA of a Targeting Molecule Folic Acid (FA) and Ethylenediamine (en)

441 mg (1 mmol) of folic acid was added to 10 ml of dry-distilled toluene, stirred for 1 hour and, then, subjected to vacuum evaporation to remove toluene and moisture. After the resulting residue was dissolved in dimethylformamide (DMF), the flask containing the mixture was soaked in an ice bath and stirred for 10 min. 226 mg (1.1 mmol) of DCC was added to the mixture and further stirred for 18 hours. 2.5 ml of ethylenediamine (en) was dissolved in 20 ml of DMF, said mixture was added thereto, and then, stirred for 3 hours. To the resulting solution was added water and acetonitrile to form a precipitate and recrystalize with the same solvent, thereby obtaining 224 mg of en-FA.

### EXAMPLE 4

### Preparation of Biocompatible and Water-Soluble Semiconductor Nanoparticles CdSe/CdS(-MPA)ₑₓ(-MUA-aPEGa)₅

0.5 ml of the CdSe/CdS(-DA)ₑₓ(-MUA)₅ solution prepared in Example 2 was diluted with chloroform to a final volume of 2.5 ml. After 6 equivalents of DCC was added to the diluent and stirred for 3 hours in a dark inert atmosphere, 7 equivalents of O,O′-Bis(2-aminoethyl)polyethylene glycol (aPEGa, MW 1,000) was added thereto and further stirred for 16 hours. The resulting solution was concentrated, mixed with acetone, and centrifuged to separate the precipitate. Thus separated precipitate was dispersed in chloroform to prepare 10 ml of a CdSe/CdS(-DA)ₑₓ(-MUA-aPEGa)₅ solution (1×10⁻⁶ M).

Subsequently, to thus prepared CdSe/CdS(-DA)ₑₓ(-MUA-aPEGa)₅ solution was added 100 equivalents of a methanol solution in which 0.05 M NaOH and 0.05 M mercaptopropionic acid (MPA) were dissolved and stirred. After the distilled water was added to the resulting solution to extract the product, a mixed solvent of methanol/ethylacetate (¼) was added thereto and centrifuged to separate the product as a precipitate. Thus separated precipitate was dispersed in a PBS buffer (pH 7.4) to prepare 10 ml of a 1×10⁻⁶ M CdSe/CdS(-MPA)ₑₓ(-MUA-aPEGa)₅ solution. The favorable bonding of aPEGa to MUA and the successful substitution of DA with MPA were confirmed by an infrared spectrophotometer and a TEM, where the results are shown in FIG. 3(e) and FIG. 4(e), respectively.

### EXAMPLE 5

### Preparation of Targeting, Biocompatible, and Water-Soluble Semiconductor Nanoparticles CdSe/CdS(-MPA)ₑₓ(-MUA-aPEGa-FA)₅ and Bio-Imaging Using the Same

2 ml of the CdSe/CdS(-DA)ₑₓ(-MUA)₅ solution prepared in Example 2 was diluted with chloroform to a final volume of 10 ml. After 6 equivalents of DCC were added to the diluent and stirred for 3 hours in a dark inert atmosphere, 15 equivalents of aPEGa-FA prepared as follows was added thereto and further stirred for 16 hours. The resulting solution was concentrated, mixed with acetone and methanol, and centrifuged to separate the precipitate. Thus separated precipitate was dispersed in chloroform to prepare 4 ml of a CdSe/CdS(-DA)ₑₓ(-MUA-aPEGa-FA)₅ solution (1×10⁻⁵ M).

Subsequently, 100 equivalents of a methanol solution prepared by dissolving 0.05 M NaOH and 0.05 M MPA was added to the CdSe/CdS(-DA)ₑₓ(-MUA-aPEGa-FA)₅ solution and stirred. After distilled water was added to the resulting solution to extract the product, a mixed solvent of methanol/ethylacetate (¼) was added thereto and centrifuged to separate the product as a precipitate. Thus separated precipitate was dispersed in a PBS buffer (pH 7.4) to prepare 10 ml of a 4×10⁻⁶ M CdSe/CdS(-MPA)ₑₓ(-MUA-aPEGa-FA)₅ solution. The favorable binding of aPEGa-FA to MUA and the successful substitution of DA with MPA were confirmed by an infrared spectrophotometer and a TEM, where the results are shown in FIG. 3(f) and FIG. 4(f), respectively.

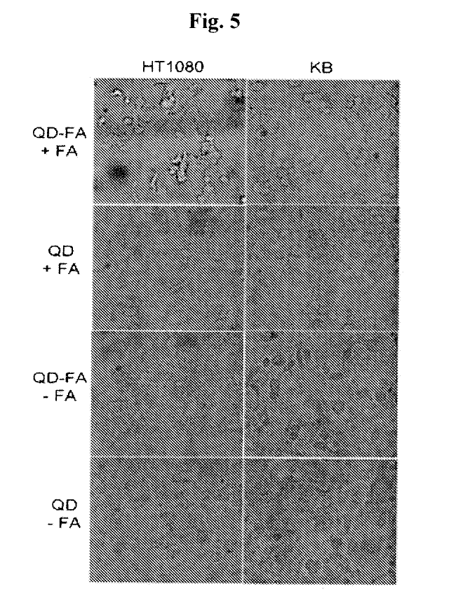

In order to examine whether the FA-bonded quantum dots CdSe/CdS(-MPA)ₑₓ(-MUA-aPEGa-FA)₅ prepared above exhibit selectivity for a certain cell line, KB cells (Human fibrosarcoma cells, ATCC, Manassas, Va.) having a folic acid receptor and HT1080 cells (Human epidermoid carcinoma cells, ATCC, Manassas, Va.) having no folic acid receptor were inoculated into the PBS solution containing FA-bonded quantum dots CdSe/CdS(-MPA)ₑₓ(-MUA-aPEGa-FA)₅ (2×10⁻⁷ M) or FA-free quantum dots CdSe/CdS(-MPA)ₑₓ(-MUA-aPEGa)₅ (2×10⁻⁷ M), in the presence or absence of free FA (9×10⁻⁶ M), respectively, and incubated at 37□ for 15 hours. After the cultivation was completed, the cells were washed with the PBS solution three times and immobilized on the surface of a slide glass. The cells were observed with a fluorescence microscope, where the results are shown in FIG. 5.

As depicted in FIG. 5, when there was no free FA in the culture solution, the quantum dots CdSe/CdS(-MPA)ₑₓ(-MUA-aPEGa-FA)₅ showing targetability were selectively transferred by a larger amount into the KB cells having a FA receptor than the quantum dots CdSe/CdS(-MPA)ₑₓ(-MUA-aPEGa)₅ showing no targetability. On the other hand, when there were abundant free FAs in the culture solution, there was no selectivity between the KB cells and HT1080 cells, since most of the free FAs bound to the corresponding receptors.

Preparation of aPEGa-FA Through the Bonding of a Biocompatible Molecule aPEGa and a Targeting Molecule FA

441 mg (1 mmol) of FA was added to 10 ml of dry-distilled toluene, stirred for 1 hour and then subjected to vacuum evaporation to remove toluene and moisture. After the resulting residue was dissolved in 14 ml of DMF, the flask containing the mixture was soaked in an ice bath and stirred for 10 min. After 226 mg (1.1 mmol) of DCC was added to the mixture and stirred for 18 hours, 1 mmol of aPEGa was added thereto and further stirred for 3 hours. Diethylether was added to the resulting solution to form a precipitate and recrystalized with the same solvent to obtain 179 mg of aPEGa-FA.

### EXAMPLE 6

### Preparation of Semiconductor Nanoparticles CdSe/CdS(-DA)ₑₓ(-MUA-aPEGa)ₙ (n=5, 10 or 30)

0.5 ml of the CdSe/CdS-DA solution prepared in Example 1 was diluted with chloroform to prepare three diluents having a final volume of 10 ml. To each diluent was added 5, 10, and 30 equivalents of MUA, respectively, and stirred for 19 hours in a dark inert atmosphere. Each solution was concentrated, mixed with methanol to form a precipitate, and then centrifuged to separate the same. Thus separated precipitate was dissolved in chloroform to prepare 2.5 ml of each CdSe/CdS(-DA)ₑₓ(-MUA)ₙ (n=5, 10, or 30) solution (4×10⁻⁶ M).

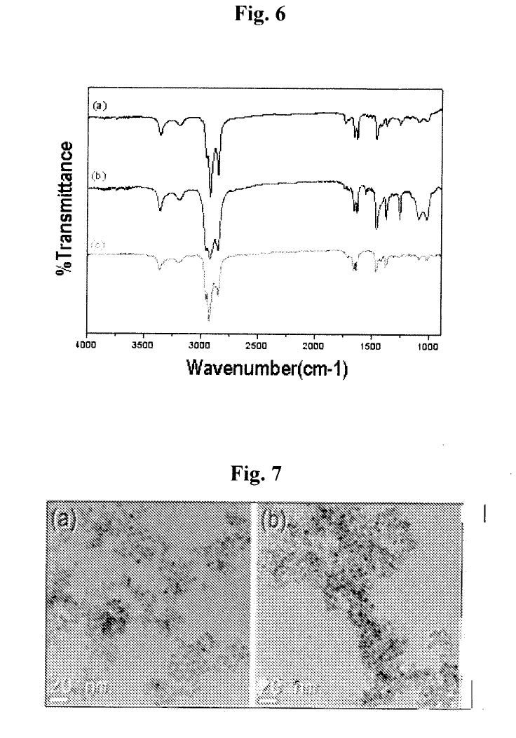

To each CdSe/CdS(-DA)ₑₓ(-MUA)ₙ (n=5, 10, or 30) solution prepared above was added 5.5, 11, and 33 equivalents of DCC, respectively, and stirred for 3 hours in a dark inert atmosphere. Subsequently, 6, 12, and 36 equivalents of aPEGa were added to each solution and further stirred for 16 hours. Each solution was concentrated, mixed with methanol to form a precipitate, and centrifuged to separate the same. Thus separated precipitate was dispersed in chloroform to prepare 10 ml of each CdSe/CdS(-DA)ₑₓ(-MUA-aPEGa)ₙ (n=5, 10, or 30) solution (1×10⁻⁶ M). It was confirmed by an infrared spectrum (FIG. 6) that, in all three cases, aPEGa successfully bound to MUA.

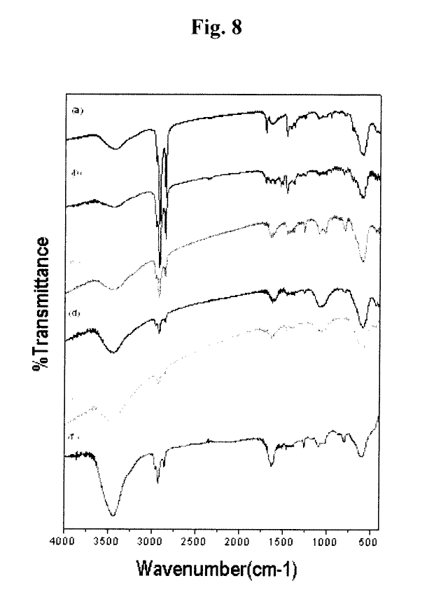

Further, it was found in the TEM image of FIG. 8 that, while the internanoparticle hydrogen bonding attraction is insignificant in the case of CdSe/CdS(-DA)ₑₓ(-MUA-aPEG)₅, in the case of CdSe/CdS(-DA)ₑₓ(-MUA-aPEG)₁₀, such effect is strong enough to induce the aggregation of nanoparticles. When n is 5 and 10 in the above formula, the nanoparticles were well dispersed in chloroform, but when n is 30, the nanoparticles formed an aggregate which was insoluble in any solvent because the internanoparticle hydrogen bonding attraction was too strong and, as a result, it was impossible to produce the TEM image of the aggregated nanoparticles. From these results, it was confirmed that, as the number of hydrophilic groups having a uniform structure at the surface of nanoparticles increases, the internanoparticle hydrogen bonding attraction can increase uncontrollably.

It was also found that there was no detectable fluorescence in the supernatant being discarded, which was obtained by adding an insoluble solvent to the quantum dot solution after reaction in Examples 1 to 6 and centrifuging the resulting solution, which suggests a 100% yield.

### EXAMPLE 7

### Preparation of Partially Surface Modified Magnetic Nanoparticles SPION(-OA)ₑₓ(-MHA)₁₀

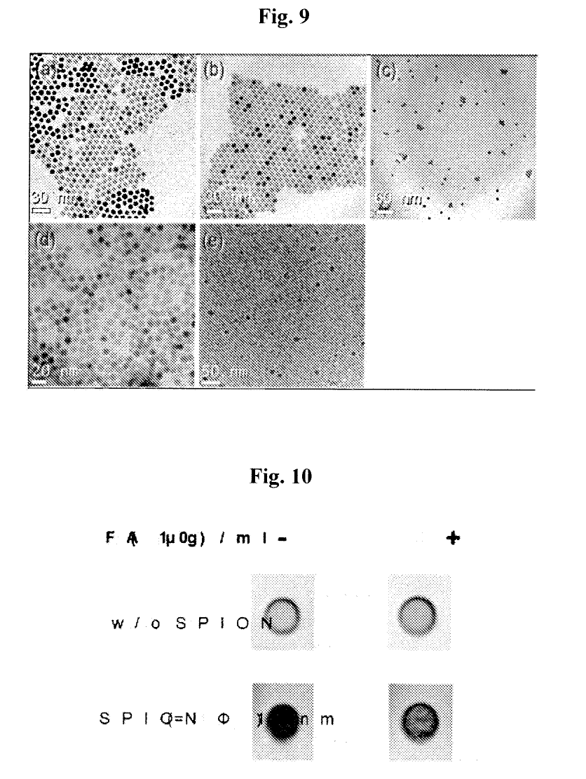

2 mg of a hydrophobic SPION-OA (SuperParamagnetic Iron Oxide Nanoparticle protected by Oleic Acid) magnetic nanoparticle solution (3×10⁻⁶ M) having a particle size of 8 nm was washed with ethanol twice, dispersed in 20 ml of toluene, and then heated to 100□. To the mixture was added 10 equivalents of mercaptohexadecanoic acid (MHA), refluxed for 1 hour, and cooled down. After concentrating 2 ml of the resulting solution, a small amount of ethanol was added thereto and, then, partially surface modified magnetic nanoparticles SPION(-OA)ₑₓ(-MHA)₁₀ were separated by using a magnet. The separated partially surface modified magnetic nanoparticles were analyzed with an infrared spectrophotometer and a TEM, where the results are shown in FIG. 8(b) and FIG. 9(b), respectively. In the TEM image, the reason the distance between the SPION(-OA)ₑₓ(-MHA)₁₀ nanoparticles is similar to that of SPION-OA was because the part of OAs are sporadically replaced with MHAs having 2 shorter carbon chains than OA, as described in step C of FIG. 2. Further, this was confirmed in the infrared spectrum that O—H peak at around 3300 cm⁻¹ is increased due to the replacement of MHA.

### EXAMPLE 8

### Preparation of Targeting, Biocompatible, and Hydrophobic Magnetic Nanoparticles SPION(-OA)ₑₓ(-MHA-aPEGa-MTX)₁₀

To 18 ml of the SPION(-OA)ₑₓ(-MHA)₁₀ solution (3×10⁻⁷ M) prepared in Example 7 was added 30 equivalents of DCC and stirred for 3 hours. After 30 equivalents of aPEGa-MTX was dissolved in a small amount of DMF as prepared as follows, the resulting solution was added to said mixture and stirred for 16 hours. After concentrating the resulting solution and mixing with ethanol, the SPION(-OA)ₑₓ(-MHA-aPEGa-MTX)₁₀ were separated by using a magnet and dispersed in 9 ml of chloroform. 1 ml of the dispersion was analyzed with an infrared spectrophotometer and a TEM, where the results are shown in FIG. 8(c) and FIG. 9(c), respectively. It was found in the TEM image that the magnetic nanoparticles broke the hexagonal arrangement due to self-assembly and were sporadically scattered, demonstrating the introduction of an irregular structure owing to aPEGa-MTX. Further, it was also confirmed in the infrared spectrum that the characteristic peaks of PEG and MTX were detected at around 1100 and 1630 cm⁻¹, respectively.

Preparation of aPEGa-MTX by Bonding a Biocompatible Molecule aPEGa to a Targeting Molecule MTX

After 454 mg (1 mmol) of MTX (methotrexate, USP reference standard) was dissolved in 14 ml of dry distillated DMF, the flask containing the mixture was soaked in an ice bath and stirred for 10 min. 226 mg (1.1 mmol) of DCC was added to the flask and stirred for 18 hours. 1 mmol of aPEGa was added thereto and further stirred for 3 hours. To the resulting solution was added diethylether to form a precipitate and recrystalized with the same solvent, thereby obtaining 138 mg of aPEGa-MTX.

### EXAMPLE 9

### Preparation of Targeting, Biocompatible, and Water-Soluble Magnetic Nanoparticles SPION(-MPA)ₑₓ(-MUA-aPEGa-MTX)₁₀

To 8 ml of the SPION(-OA)ₑₓ(-MHA-aPEGa-MTX)₁₀ solution (6×10⁻⁷ M) prepared in Example 8 was added 150 equivalents of a methanol solution prepared by dissolving 0.05 M MPA and 0.05 M NaOH and stirred for 2 hours. Methanol was added to the resulting solution and centrifuged to obtain solid SPION(-MPA)ₑₓ(-MUA-aPEGa-MTX)₁₀. The SPION(-MPA)ₑₓ(-MUA-aPEGa-MTX)₁₀ was dispersed in 16 ml of a PBS buffer (pH 7.4). 1 mg of the dispersion was analyzed with an infrared spectrophotometer and a TEM, where the results are shown in FIG. 8(d) and FIG. 9(d), respectively. It was found in the infrared spectrum that the intensity of C—H peak was significantly decreased since the length of the carbon chain was decreased as OA was replaced with MPA. Further, it was confirmed in the TEM image that, in spite of an overlapping effect due to surface tension caused by using an aqueous solution when preparing the sample, the interparticle distance was clearly observed.

### EXAMPLE 10

### Preparation of Targeting, Biocompatible, and Water-Soluble Magnetic Nanoparticles SPION(-Lys)ₑₓ(-MUA-aPEGa-MTX)₁₀

To 6 ml of the SPION(-OA)ₑₓ(-MHA-aPEGa-MTX)₁₀ solution (6×10⁻⁷ M) prepared in Examples 7 and 8 was added 1,000 equivalents of tetraoctylammonium bromide (TOAB) and stirred for 16 hours. 6 ml of a 0.1 M lysine (Lys) aqueous solution was added to the mixture and stirred for 19 hours. The resulting solution was mixed with methanol and SPION(-Lys)ₑₓ(-MUA-aPEGa-MTX)₁₀ nanoparticles were separated by using a magnet. The separated nanoparticles were washed with 5 ml of toluene and 5 ml of ethanol, and dispersed in a PBS buffer (pH 7.4). 1 ml of the dispersion was analyzed with an infrared spectrophotometer and a TEM, where the results are shown in FIG. 8(e) and FIG. 9(e), respectively. It was found in the infrared spectrum that the intensity of C—H peak was significantly decreased since the length of a carbon chain was decreased as OA was replaced with lysine. Further, it was also confirmed in the TEM image that, in spite of an overlapping effect due to the surface tension caused by using an aqueous solution when preparing the sample, the interparticle distance was clearly observed.

### EXAMPLE 11

### Preparation of Targeting, Biocompatible, and Hydrophobic Magnetic Nanoparticles SPION(-OA)ₑₓ(-MHA-en-FA)₅ and Bio-Imaging Using the Same

2 ml of the hydrophobic SPION-OA magnetic nanoparticle solution (3×10⁻⁶ M) having a particle size of 11 nm was washed with ethanol twice, dispersed in 20 ml of toluene, and then heated to 100□. 5 equivalents of mercaptohexadecanoic acid (MHA) was added to the mixture, refluxed for 1 hour, and cooled down. To the resulting solution was added 15 equivalents of DCC and stirred for 3 hours in a dark inert atmosphere. 16 equivalents of en-FA prepared in Example 13 prepared by dissolving in a small amount of DMF was added thereto and stirred for 16 hours. After concentrating the resulting solution, a small amount of ethanol was added thereto and SPION(-OA)ₑₓ(-MHA-en-FA)₅ nanoparticles were separated by using a magnet. The separated nanoparticles were dispersed in 10 ml of chloroform. The infrared spectrum of these nanoparticles is shown in FIG. 8(f), where it was confirmed that a characteristic peak of FA is detected at around 1630 cm⁻¹.

After the SPION(-OA)ₑₓ(-MHA-en-FA)₅ solution prepared above was diluted to a final concentration of 1×10⁻⁷ M, 1 ml of the diluent was uniformly spread onto a culture dish and naturally dried. The next day, the culture dish was sterilized with 70% ethanol and UV, 2×10⁵ cells of human epithelial cancer cell line KB cells (ATCC Manassas, Va.) were inoculated, and then the cells were cultured in a 37□ incubator for 16 hours. The cultured cells were collected and further cultured in an untreated culture dish for 4 hours. In order to compare the targeting behavior of the nanoparticles, the same experiment was conducted as described above, except that 10 μg/ml of FA was added to the culture solution. Each of thus treated cells was analyzed with magnetic resonance imaging (MRI), where the results are shown in FIG. 10. As a result of quantitatively analyzing the MR image, it was confirmed that the coexisting excess free FAs occupy the receptors on the cell surface, resulting in about a 10% reduction in targetability of the targeting magnetic nanoparticles SPION(-OA)ₑₓ(-MHA-en-FA)₅. These results suggest that the hydrophobic magnetic nanoparticles exhibit significantly meaningful targetability by a bonded FA.

While the present invention has been described and illustrated with respect to a preferred embodiment of the invention, it will be apparent to those skilled in the art that variations and modifications are possible without deviating from the broad principles and teachings of the present invention, which should be limited solely by the scope of the claims appended hereto.

1. A method of preparing bio-imaging nanoparticles which comprises:
1) adding 1 to 30 equivalents of an organic ligand, which contains a thiol group and a hydrophilic group being linked by a hydrocarbon chain of from 8 to 20 carbon atoms, to core or core/shell hydrophobic inorganic nanoparticles protected with surfactants, under conditions effective to partially replace the surfactants with organic ligands and thereby form a metal-thiolate (M-S) bond on the surface of nanoparticles, resulting in preparing hydrophobic nanoparticles whose surface is only partially modified to hydrophilic, while still maintaining their individual dispersibility in a nonpolar organic solvent;
2) bonding functional molecules to the hydrophilic groups introduced onto the surface of nanoparticles prepared in step 1), to afford functionality and an irregular structure to the surface of nanoparticles, while maintaining their individual dispersibility; and
3) replacing the rest of the surfactants remaining on the surface of nanoparticles prepared in step 2) with organic ligands where at least two hydrophilic groups are linked by a hydrocarbon chain of from 1 to 7 carbon atoms, thereby converting the hydrophobic nanoparticles into hydrophilic ones.

2. The method according to claim 1, wherein the inorganic nanoparticle of step 1) is a noble metal nanoparticle, an iron oxide nanoparticle, or a semiconductor nanoparticle comprising of an element belonging to family II of a periodic table selected from the group consisting of zinc, cadmium, and lead, and another element belonging to family VI of a periodic table selected from sulfur, selenium, and tellurium.

3. The method according to claim 2, wherein the inorganic nanoparticle is selected from the group consisting of CdSe, ZnS, CdSe/CdS, CdSe/ZnS, Au, Ag, Fe₂O₃ and Fe₃O₄.

4. The method according to claim 1, wherein the organic ligand of step 1) comprises at least one thiol group and at least one hydrophilic group within the ligand molecule, wherein the hydrophilic group is selected from the group consisting of an amine group, a carboxylic acid group, a hydroxy group, and a thiol group.

5. The method according to claim 4, wherein the organic ligand of step 1) is selected from the group consisting of mercaptohexadecanoic acid, mercaptododecanoic acid, and mercaptohexadecanoic acid.

6. The method according to claim 1, wherein the number of organic ligands in step 1) capable of binding to the surface of nanoparticles via a metal-thiolate (M-S) covalent bond is in the range of from 1 to 30.

7. The method according to claim 1, wherein the functional molecule of step 2) is a biocompatible molecule, a targeting molecule, a complex thereof, or a mixture thereof, which contains a hydrophilic group selected from the group consisting of an amine group, an aldehyde group, a carboxylic acid group, a hydroxy group, and a thiol group being linked to one or both terminal ends thereof.

8. The method according to claim 1, wherein the organic ligand of step 3) contains at least two hydrophilic groups, wherein the hydrophilic group is selected from the group consisting of an amine group, a carboxylic acid group, a hydroxy group, and a thiol group.

9. The method according to claim 8, wherein the organic ligand of step 3) is selected from the group consisting of mercaptohexanoic acid, mercaptoacetic acid, mercaptopropionic acid, dimercaptosuccinic acid, 2-mercaptoethanol, 2-aminoethanethiol, lysine, arginine, and aminovaleric acid.

10. The method according to claim 7, wherein the biocompatible molecule contains a hydrophilic group selected from the group consisting of an amine group, an aldehyde group, and a carboxylic acid group at both terminal ends, or contains a hydrophilic group selected from the group consisting of an amine group, an aldehyde group, and a carboxylic acid group at one terminal end and an alkoxyl group or a hydroxy group having 1 to 7 carbon atoms at the other terminal end.

11. The method according to claim 10, wherein the biocompatible molecule is selected from the group consisting of polyethylene glycol (PEG), dextran, poly(L-lactide) (PLLA), poly(DL-lactide) (PDLLA), poly-DL-lactide/glycolide copolymer (PLGA), chitosan, alginic acid, hyaluronic acid, collagen, heparin, and poly(ε-caprolacton).

12. The method according to claim 7, wherein the targeting molecule can be specifically recognized in vivo, contains a hydrophilic group selected from the group consisting of an amine group, a carboxylic acid group, a hydroxy group, and a thiol group, and can be linked to a biocompatible molecule through the formation of an amide bond, an ester bond, or a thioester bond.

13. The method according to claim 12, wherein the targeting molecule is folic acid, methotrexate (MTX), a peptide selective for a specific cell, or an antibody selectively reacting with a specific antigen.

14. Bio-imaging nanoparticles prepared in step 1) of the method according to claim 1, which are hydrophilic only at the part of the nanoparticle surface wherein organic ligands containing a thiol group and a hydrophilic group being linked by a hydrocarbon chain of from 8 to 20 carbon atoms are introduced into the surface of core or core/shell hydrophobic nanoparticles protected with surfactants, but are still hydrophobic as a whole, and maintain their individual dispersibility in a nonpolar organic solvent.

15. Bio-imaging nanoparticles prepared in step 2) of the method according to claim 1, which have functionality and an irregular structure due to the bonding of functional molecules to the hydrophilic groups introduced into the surface of nanoparticles according to claim 14, and are hydrophilic only at the part of the nanoparticle surface wherein the functional molecules bind to, but are still hydrophobic as a whole.

16. Bio-imaging nanoparticles prepared in step 3) of the method according to claim 1, which are completely converted into hydrophilic nanoparticles by replacing the rest of the surfactants remaining on the surface of nanoparticles according to claim 15 with organic ligands containing at least two hydrophilic groups being linked by a hydrocarbon chain of from 1 to 7 carbon atoms, and maintain their individual dispersibility in an aqueous solution.
